(19) United States
(12) Patent Application Publication
(10) Pub. No.: US 2007/0066863 A1
(43) Pub. Date: Mar. 22, 2007

(54) DEVICE FOR TREATING MITRAL VALVE REGURGITATION

(75) Inventors: Nasser Rafiee, Andover, MA (US); Eliot F. Bloom, Hopkington, NH (US); Raymond Godaire, Auburn, MA (US); Michael Finney, Beverly, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/468,844

(22) Filed: Aug. 31, 2006

Related U.S. Application Data
(60) Provisional application No. 60/713,299, filed on Aug. 31, 2005.
(60) Provisional application No. 60/743,349, filed on Feb. 24, 2006.

(51) Int. Cl. A61F 2/02; A61F 2/24

(52) U.S. Cl. 600/37; 606/151; 623/2.36

(57) ABSTRACT

A system for treating mitral valve regurgitation comprises tensioning device that can be deployed using a delivery catheter. The device includes tension member linking a proximal anchor and distal anchor. The device is constructed from a material having suitable elastic properties such that the device applies a constant tension force between the anchors, while stretching or flexing in response to a heartbeat when positioned across a chamber of a heart. The anchors may include a plurality of arms. In some embodiments, the arms may also flex in response to a heart beat. When positioned across the left ventricle of a heart, the device can reduce the lateral distance between the walls of the ventricle and thus allow better coaption of the mitral valve leaflets thereby reducing mitral regurgitation.

### PRIORITY CLAIM

This application claims priority under 35 U.S.C. §119(e) from U.S. Provisional Application No. 60/713,299 filed Aug. 31, 2005; and U.S. Provisional Application No. 60/743,349, filed Feb. 24, 2006; the entirety of each of which is hereby incorporated by reference thereto

### TECHNICAL FILED

This invention relates generally to medical devices and particularly to a system and method for treating mitral valve regurgitation.

### BACKGROUND OF THE INVENTION

The heart is a four-chambered pump that moves blood efficiently through the vascular system. Blood enters the heart through the vena cava and flows into the right atrium. From the right atrium blood flows through the tricuspid valve and into the right ventricle, which then contracts and forces blood through the pulmonic valve and into the lung. Oxygenated blood returns from the lungs and enters the heart through the left atrium and pas s through the bicuspid mitral valve into the left ventricle. The left ventricle contracts and pumps blood through the aortic valve into the aorta and to the vascular system.

The mitral valve consists of two leaflets (anterior and posterior) attached to a fibrous ring or annulus. In a healthy hearts the mitral valve leaflets overlap during contraction of the left ventricle and prevent blood from flowing back into the left atrium. However, due to various cardiac diseases, the mitral valve annulus may become distended, causing the leaflets to remain partially open during ventricular contraction and thus allowing regurgitation of blood into the left atrium. This results in reduced ejection volume from the eft ventricle, causing the left ventricle to compensate with a larger stroke volume. The increased workload eventually results in dilation and hypertrophy of the left ventricle, further enlarging and distorting the shape of the mitral valve. If left untreated, the condition may result in cardiac insufficiency, ventricular failure, and death.

it is common medical practice to treat mitral valve regurgitation by valve replacement or repair. Valve replacement involves an open-heart surgical procedure in which the patient's mitral valve is removed and replaced with an artificial valve. This is a complex, invasive surgical procedure with the potential for many complications and a long recovery period.

Mitral valve repair includes a variety of procedures to repair or reshape the leaflets to improve closure of the valve during ventricular contraction. If the mitral valve annulus has become distended, a common repair procedure involves implanting an annuloplasty ring on the mitral valve annulus. The annuloplasty ring generally has a smaller diameter than the annulus, and when sutured to the annulus, the annuloplasty ring draws the annulus into a smaller configuration, bringing the mitral valve leaflets closer together and providing improved closure during ventricular contraction.

Annuloplasty rings may be rigid, flexible, or have both rigid and flexible segments. Rigid annuloplasty rings have the disadvantage of causing the mitral valve annulus to be rigid and unable to flex in response to the contractions of the ventricle, thus inhibiting the normal movement of the mitral valve that is required for it to function optimally. Flexible annuloplasty rings are frequently made of Dacron® fabric and must be sewn to the annular ring with a line of sutures. This eventually leads to scar tissue formation and loss of flexibility and function of the mitral valve. Similarly, combination rings must generally be sutured in place and also cause scar tissue formation and loss of mitral valve flexibility and function.

Annuloplasty rings have been developed that do not require suturing, U.S. Pat. No. 6,565,603 discloses a combination rigid and flexible annuloplasty ring that is inserted into the tat pad of the atrioventricular groove, which surrounds the mitral valve annulus. Although this device avoids the need for sutures, it must be placed within the atrioventricular groove with great care to prevent tissue damage to the heart.

U.S. Pat. No. 6,569,198 discloses a flexible annuloplasty ring designed to be inserted into the coronary sinus, which is located adjacent to and partially surrounds the mitral annulus. The prosthesis is shortened lengthwise within the coronary sinus to reduce the size of the mitral annulus. However, the coronary sinus in a particular individual may not wrap around the heart far enough to allow effective encircling of the mitral valves making this treatment ineffective.

U.S. Pat. No. 6,210,432 discloses a flexible elongated device that is inserted into the coronary sinus and adapts to the shape of the coronary sinus. The device then undergoes a change that causes it to assume a reduced radius of curvature and, as a result, causes the radius of curvature of the coronary sinus and the circumference of the mitral annulus to be reduced. While likely to be effective for modest changes in the size or shape of the mitral annulus, this device may cause significant tissue compression in patients requiring a larger change in the configuration of the mitral annulus.

U.S. Pat. No. 6,908,478 discloses a flexible elongated device that is inserted into the coronary sinus and anchored at each end by a self-expanding, toggle bolt-like anchor that expands and engages the inner wall of the coronary sinus. Application WO02/076284 discloses a similar flexible elongated device that is inserted into the coronary sinus. This device is anchored at the distal end by puncturing the wall of the coronary sinus, crossing the intervening cardiac tissue, and deploying the anchor against the exterior of the heart in the pericardial space. The proximal end of the elongated member is anchored against the coronary ostium, which connects the right atrium and the coronary sinus. Once anchored at each end, the length of either of the elongated devices may be adjusted to reduce the curvature of the coronary sinus and thereby change the configuration of the mitral annulus. Due to the nature of the anchors, both of these devices may cause significant damage to the coronary sinus and surrounding cardiac tissue. Also, leaving a device in the coronary sinus may result in formation and breaking off of a thrombus that may pass into the right atrium, right ventricle, and ultimately the lungs, causing a pulmonary embolism. Another disadvantage is that the coronary sinus is typically used for placement of a pacing lead, which may be precluded with the placement of the prosthesis in the coronary sinus.

U.S. Pat. No. 5,961,440 (the contents of which are incorporated into this section by reference thereto) discloses a method for calculating the tension in the walls of a heart chamber (column 10, line 25 to column 11, line 7). It has also been stated that one can calculate the wall stiffness (in the walls of a heart chamber) by calculating the change in chamber volume over the change in pressure within the chamber (ΔV/ΔP).

U.S. Pat. No. 6,616,684 (the '684 patent) discloses splint assemblies that are positioned transverse the left ventricle to reduce tension in the walls of a heart chamber, thereby reducing mitral valve leakage. In one embodiment, the assembly is delivered through the right ventricle. One end of the assembly is anchored outside the heart, resting against the outside wall of the left ventricle, while the other end is anchored within the right ventricle, against the septal wall. The heart-engaging portions of the assembly, i.e., the anchors, are essentially flat and lie snugly against their respective walls. The length of the splint assembly is either preset or is adjusted to draw the two walls of the chamber toward each other.

The splint assembly may be delivered endovascularly, which offers distinct advantages over open surgery methods. First, a puncture device is delivered into the right ventricle, advanced through the septal wall, and anchored to the outer or free wall of the left ventricle using barbs or threads that are rotated into the tissue of the free wall. A delivery catheter is then advanced over the needle, piercing both the septal wall and the free wall of the ventricle. A tension member is then pushed through the delivery catheter such that a distal anchor is positioned outside the heart. The catheter is withdrawn, and a second (proximal) anchor is advanced over the tension member using a deployment tool and positioned within the right ventricle against the septal wall. A tightening device then holds the second anchor in a position so as to after the shape of the left ventricle. Excess length of the tension severed prior to removal. Another device for shortening the distance between the septal wall of a heart chamber and the free wall of the chamber can be see in the published U.S. Patent Application No. 2004/0260317, the contents of which are incorporated herein by reference.

One potential problem encountered when using devices of the type described in the '684 patent is that oversized anchors could tend to negatively alter the chamber geometry and undersized anchors may migrate through the heart tissue due to forces exerted by a beating heart. Both the '684 patent and U.S. Pat. No. 6,537,198 disclose addressing this by property dimensioning the anchors for the splint devices disclosed therein. However, it would be desirable to have devices that addressed the issue of anchor migration in a manner other than strict dimensioning of the anchors. Such devices could then be used to treat a variety of heart sizes and they may even allow treatment on hearts having injured tissue that may be more susceptible to anchor migration.

Therefore, it would be desirable to provide a system and method for treating mitral valve regurgitation that overcome the aforementioned and other disadvantages.

### SUMMARY OF THE INVENTION

One aspect of the present invention is a device for treating mitral valve regurgitation, comprising a tension member and proximal and distal anchors. The distal anchor is attached to a distal end of the tension member, and the proximal anchor is attached to a proximal and of the tether.

As used herein, the terms “distal” and “proximal” refer to the location of the referenced element with respect to the treating clinician during deployment of the device with proximal being closer to the treating clinician than distal. Additionally, when used to describe the devices herein, the term “elastic,” or variations thereof, shall be understood to mean the ability to stretch/distort from a first length to a send length under a force/load and then return to the first length when the force/load is removed. Similarly, the term “flexible” or variations thereof shall be understood to mean the ability to distort/flex from a first shape to a second shape under a force/load and then return to the first shape when the force/load is removed Various embodiments of the devices of the current invention may be referred to herein simply as “the device” or the “tensioning device” and both terms are to be understood to mean the same thing herein.

The devices described herein comprise a biocompatible material capable of being preset into a desired shape. Such materials should be sufficiently elastic and flexible that the tension member applies a constant tension force between the anchors, while flexing and/or stretching in response to a heartbeat when the device is positioned across a chamb of a heart. In some embodiments, the devices can be constructed from wires of such materials or braided from such materials, and in others the device can be cut from tubes of such materials.

Another aspect of the present invention is a system for treating mitral valve regurgitation that includes the above-described tensioning device and further comprises a delivery catheter. The device is slidably received within a lumen of the delivery catheter.

Another aspect of the present invention is a method of treating mitral valve regurgitation by affecting a mitral valve annulus. A first wall of a chamber of a heart is pierced. A distal anchor is engaged with a second wall of the heart chamber. A proximal anchor is engaged with the first wall of the heart chamber. A tension member links the proximal and distal anchors applies a constant tension force to reduce the lateral distance between the two anchors.

Devices disclosed herein are advantageous over previously disclosed devices in that they dampen shock to supporting tissues and have reduced fatigue relative to other devices, there is no stack state in which a chord or tensioning member may float around inside of a heart chamber, the devices can be easier to deploy than previously disclosed devices, and they can provide a reduction in thrombus formation compared to previously disclosed devices. Another advantage over previously disclosed devices is that the devices disclosed herein are made to be the appropriate length before they are installed, thus there is no adjustment required when the device is implanted in a heart.

The aforementioned and other features and advantages of the invention will become further apparent from the following detailed description, read in conjunction with the accompanying drawings, which are not to scale. The detailed description and drawings are not scale, and should be viewed as being merely illustrative of the invention rather than limiting, the scope of the Invention being defined by the appended claims and equivalents thereof.

### BRIEF DESCRIPTION OF THE DRAWINGS

FIG. 1 is a side view of one embodiment of a device for treating mitral regurgitation in accordance with the present invention;

FIG. 2 is a side view of the device shown in FIG. 1 in an elongated state;

FIGS. 3-5 are side views of additional embodiments of devices for treating mitral regurgitation as described herein;

FIG. 5A is a partial schematic view illustrating the placement of an anchor shown in FIG. 5;

FIG. 6 is an isometric view of an embodiment of a device for treating mitral regurgitation in accordance with the present invention, shown in the context of a system for treating mitral valve regurgitation in accordance with the present invention, the tensioning device being completely shown, and a guiding catheter being shown in cross section;

FIGS. 7 & 8 are schema tic views illustrating the placement of the tensioning device across a left ventricle, in accordance with the present invention;

FIG. 9 is an ideal response curve, for the elongation under load, of materials used for the devices disclosed herein; and

FIG. 10 is a flow diagram of one embodiment of a method for treating mitral valve regurgitation in accordance with the present invention.

### DESCRIPTION OF THE INVENTION

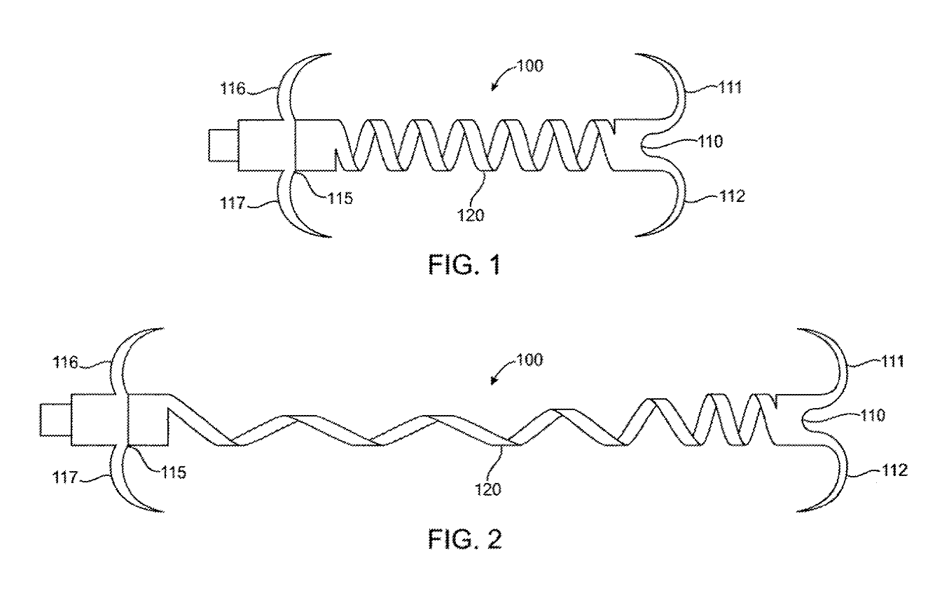

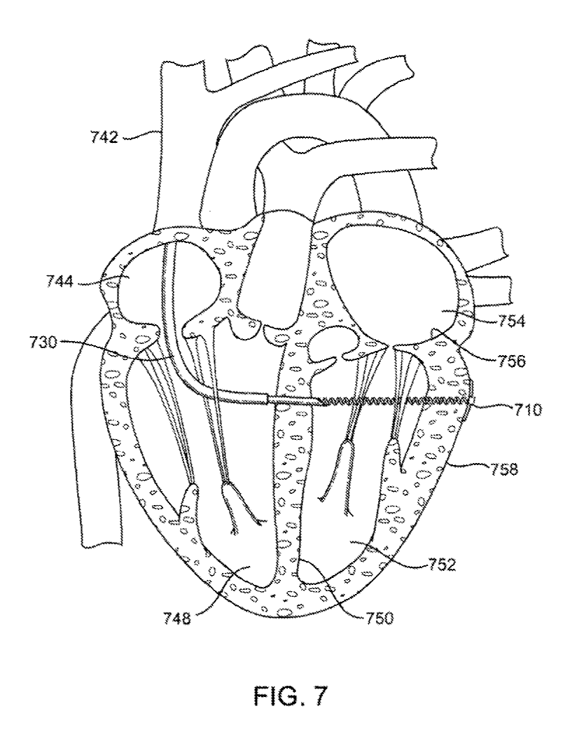

The invention will now be described in detail below by referring to the attached drawings, where like numbers refer to like structures. One aspect of the present invention is a device for treating mitral valve regurgitation by reducing the lateral space between the septal wall and the free wall of a heart chamber. One embodiment of a device, in accordance with the present invention, is illustrated in FIGS. 1 & 2, which show the device in a deployed/deployment configuration as opposed to a delivery configuration as depicted in FIG. 6.

Tensioning device 100 is designed to be positioned across a chamber of a heart so that it reduces the distance between the septum and the free wall of a heart chamber to alter the chamber geometry and wall tension thereby reducing valvular regurgitation. Although described below in the context of treating mitral valve regurgitation by reducing or limiting lateral distension of the left ventricle as the heart beats, device 100 may be deployed at other locations in the heart and is readily adapted to a wide variety of uses, including treating ischemic or dilated cardiomyopathy.

As can be seen in FIG. 1, the device 100 includes a proximal anchor 115 positioned adjacent to the proximal end of a tension member 120, and a distal anchor 110 positioned adjacent to the distal end of the tension member 115. The device is constructed from an elastic material such that the tension member 120 becomes elongated (stretches) in response to a heartbeat when the device 100 is positioned across a heart chamber. The embodiment shown in FIG. 1 is a helical extension spring constructed cut from a tube of material having the desired elasticity.

Prior to implantation, a clinician can determine the desired length of the tension device by determining the distance between the septum and the free wall, the desired wall tension of the heart chamber, and the desired distance between the septum and the free wall using the formulas noted in the background section of this document and standard visualization techniques. Once desired length for the device and the desired wall tension/stiffness for the walls of the heart chamber is determined, a spring constant for the tension member can be determined for minimum spring compliance such that the tension member will slightly stretch when the heart beats but the device will not become too elongated.

After the desired spring compliance and spring constant are determined, the spring is designed so that it has the minimum physical dimensions possible that would allow it to perform as desired. Thus, a clinician would want a spring having a minimum outer diameter. For helical coil type springs, a clinician would try to achieve the minimum material diameter of the material used to make the coils and the minimum number of coils per Inch needed for the spring to perform as desired.

It should be noted that the spring constant (k) is determined using Hooke's Law (F=kx, where F=Force and x=spring displacement) and the spring compliance is the reciprocal of the spring constant (compliance=1/k). One embodiment of the current invention has a compliance of five-percent (5%), while another embodiment of the invention has a spring compliance of twenty-percent (20%), and other embodiments of the invention can have spring compliancies in the range of one-peroent (1%) to twenty-five-percent (25%).

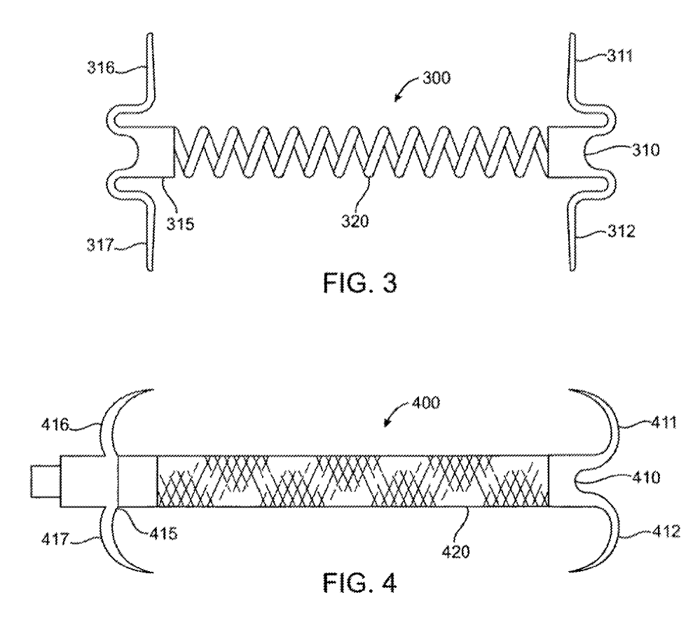

FIG. 2 depicts the device with the tension member in an elongated state. The state of elongation is exaggerated, relative to actual elongation when implanted in a heart chamber, to illustrate the fact that the length of the tension member can change under load. When the device is implanted in a heart chamber, the tension member 120 will become sightly elongated as the force load on the member is increased during diastole and it will contract to its original shape when the force load decreases during systole. The distal and proximal anchors of the device 110 & 115 respectively, each have a plurality of extendable arms 111, 112, 117, & 117. In at least one embodiment of the invention, the arms of at least one of the anchoring sections will also flex in response to a heart boat to further reduce the stress caused by the anchors on the chamber walls. Elastic stretching of the tension member with or without additional flexing of anchor arms, reduces the risk of the device failing due to structural fatigue, and also reduces localized compressive pressure on tissue against which the anchors rest thereby reducing the potential for anchor migration through the heart chamber walls.

In the various embodiments described herein, the device comprises a suitable biocompatible material- Such materials include, but, are not limited to, a nickel-titanium alloy, a nickel-cobalt alloy, other cobalt alloys, a thermoset plastics a thermoplastic, stainless steel, a suitable biocompatible shape-memory material, a suitable biocompatible super elastic material, combinations thereof, and the like In some embodiments the devices can be constructed from wares of such materials and in others; the devices can be braided from such materials. In at least one embodiment, the device is cut from a tube of such materials. The cross-sectional shape of the coils of such devices can vary based on the characteristics of the materials and in at least one embodiment the transverse cross-sectional shape of the material used to make the spring coils is round.

In the embodiments depicted in FIGS. 1 through 4, each of proximal and distal anchors comprises a hub portion having evenly flexible arms formed therefrom. While these figures show the devices in side view such that only two arm sections are seen, the embodiments of devices shown have three arms per anchor. In other embodiments, the arms may be formed separately from the body of the anchor and assembled to create an integral whole. Additionally, the anchors of various embodiments of the invention can Include two, three, tour, or more arms.

During manufacture of at least one embodiment, the arms are bent outward and heat set or otherwise set such that each of the arms is self-deploying radially outward at an angle of between 40 and 90 degrees from the longitudinal axis of the anchor when the anchor is released from a delivery catheter. The length of the arms is generally calculated so that the diameter of a circle that would just cover the expanded anchor is five times the diameter of the piercing tube/catheter (634 of FIG. 6) that is used to deliver the device to a heart chamber. FIG. 6 shows an embodiment of a tensioning device 600 in a delivery configuration. The delivery configuration of the device having distal and proximal anchors each with four flexible arms in a radially compressed, folded configuration while the anchors are within delivery catheter 630. When the device s deployed from the deliver catheter, the arms wilt self expand to a deployed configuration (as shown in FIGS. 1 & 2).

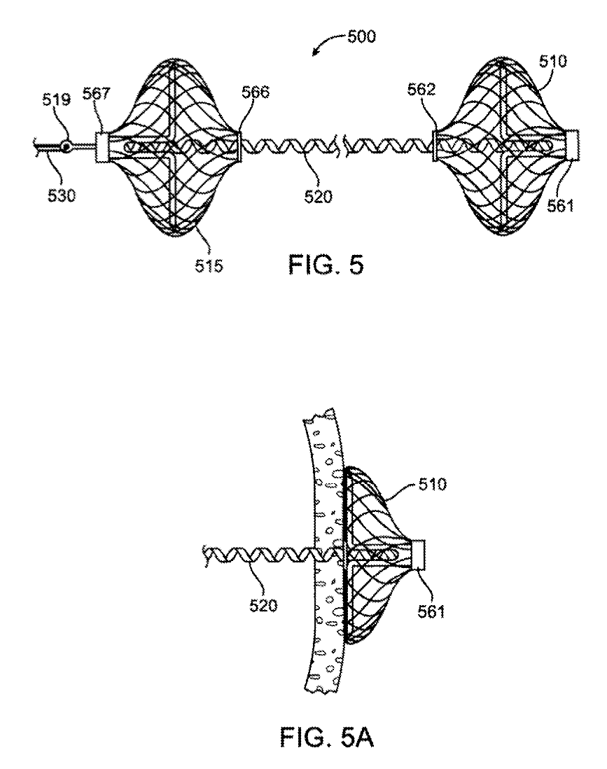

FIGS. 3 & 4 depict alternate embodiments of the tensioning devices disclosed herein. The device 300 depicted in FIG. 3 has a tension member 320 configured in a helical spring shape and formed from wire or the like. The device includes a proximal anchor 315 positioned adjacent to the proximal end of a tension member 320, and a distal anchor 315 6positioned adjacent to the distant end of the tension member 315. Both the proximal and distal anchor members have a plurality of arm segments 311, 312, 316, & 317 for resting against a heart chamber wall. At least one embodiment of the current invention having arms similar to those shown in FIG. 3, includes flexible arms.

The device 400 depicted in FIG. 4 has a tension member made from some material of suitable elastic properties but not having a helical spring type configuration. Embodiments similar to the depicted embodiment can be made from elastic monofilament, braided elastomeric thread, or the like. The device includes a proximal anchor 415 positioned adjacent to the proximal end of a tension member 420, and a distal anchor 410 positioned adjacent to the distal end of the tension member. Both the proximal and distal anchor members have a plurality of arms 411, 412, 416, & 417 for resting against a heart chamber wall, and the arms can be made from a suitable flexible material.

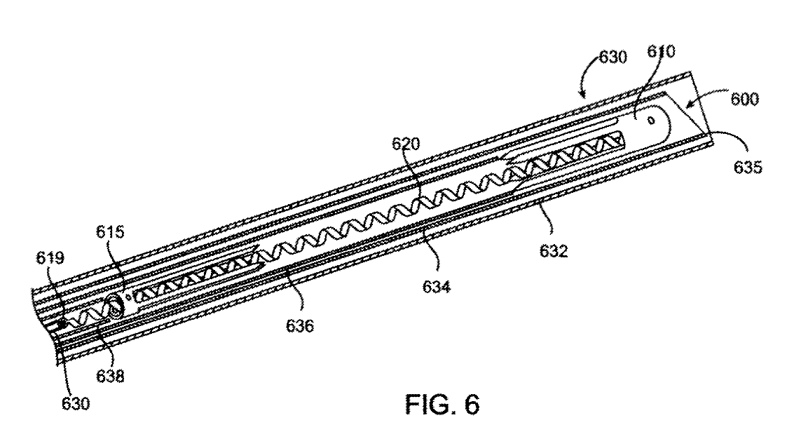

FIG. 5 depicts another embodiment of the current invention. As can be seen in FIG. 5, the device 500 includes distal and proximal anchors 510, 515 having portions that are made from a plurality of fibers braided into a tubular configuration. The tubular braid anchors can be made from fibers comprising any biocompatible material that will provide suitable strength and flexibility. The tubular braided portion of the anchors for the embodiment depicted in FIG. 5 surround anchor sections with a plurality of arms similar to the arms of the anchors shown in the embodiments of the invention depicted in FIGS. 1-4.

The tubular braided poton of the distal anchor 510 includes a fixed hub 561 that is attached to the distal portion of the tension member and an inside hub 562 that moves freely along the length of the helical spring tension member 520. The proximal anchor 515 includes a fixed hub 567 that is attached to the proximal portion of the tension member and an inside hub 566 that moves freely along the length of the tension member. An anchor adjustment chord 530 is routed through an eyelet 519 at the proximal most end of the tension member and back through a delivery catheter (not shown) to a clinician. While not depicted with the devices shown in FIGS. 1-4, embodiments of devices similar to those depicted do have an eyelet similar to eyelet 519 shown in FIG. 5. When the tension device 500 is deployed, a clinician can apply tension to the adjustment cord to keep the proximal end of the device in the delivery catheter and keep the proximal anchor from deploying within the heart chamber.

According to the current invention, there are a plurality of ways to make the anchors assume a deployed configuration after delivery. In one preferred embodiment, the anchors can be made from a shape memory material and then pre-set in a deployment configuration before being forced into, and restrained in, a delivery configuration. In other embodiments of tension devices the anchors can be mechanically forced into the deployment configuration after delivery to a heart chamber.

Referring to FIG. 5A, when the device depicted in FIG. 5 is deployed and tension is applied, the arms of the anchor extend outward to a deployment configuration while the center of the braided portions expand radially to an essentially circular disc around the arms. The figure shows the anchor in a slightly domed state just as tension is starting to be applied. As the device comes under the full tension load, the anchors are pulled flat into a generally flat disc. Thus the arms provide support for the anchor against the walls of the chamber. The combination of the arms and the braided portion allows for an anchor that can rest solidly against the walls of the heart and not migrate through the chamber walls.

The anchors can be configured for catheter delivery to a ventricle and then expanded to a generally planar deployment configuration to rest against the septum or free wall of a hear. In a delivery configuration, the tubular braided anchors have a relatively small outer diameter to allow them to pass through a delivery catheter or other delivery member. Once the anchors are deployed, they can assume a deployment configuration where a portion of the tubular braid expands radially outward such that the deployed anchor has a larger outside diameter than it had in a delivery configuration.

Referring now to FIG. 6, there is shown an embodiment of a tensioning device (shown generally as 600) as disclosed herein, that is slidably received within a lumen of delivery catheter 630 for delivery to and deployment at the treatment area. The delivery catheter 630 comprises a guiding sheath 632, a piercing tube 634 having a beveled portion 635, a holding tube 636, and a push cylinder 638. Piercing tube 634 is slidable within a lumen of guiding sheath 632, holding tube 636 is slidable within a lumen of piercing tube 634, and push cylinder 638 is slidable within a lumen of holding tube 636. Thus, delivery catheter 630 comprises four separate, concentric members, each slidable to be individually extended or retracted as needed to deliver the device 600.

The device embodiment 600 depicted in FIG. 6 has a tension member 620 with a distal anchor 610 and a proximal anchor 615. Each anchor has four flexible arms that are collapsed into a delivery configuration and the arms expand into a deployment configuration when the tensioning device is expelled from the delivery catheter. The device includes an adjustment member 630 that is routed through an eyelet 619 at the proximal end of the tension member. During deployment of the device, the clinician can use the adjustment member to keep the proximal anchor from entering the ventricle when the device is being deployed.

Guiding sheath 632 comprises a flexible, biocompatible material such as polyurethane, polyethylene, nylon, fluoropolymers, or the like Guiding sheath 632 has a preformed or steerable distal tip that is capable of assuming a desired bend with respect to the longitudinal axis of the sheath, for example, a ninety-degree bend.

Piercing tube 634 comprises the same or a different biocompatible material from that used to form guiding sheath 632. In the present embodiment, the distal end of piercing tube 634 is angle-cut to form a sharp edge 635 able to pierce into or through myocardial tissue. Thus, where a device of the current invention is to be delivered transturminaily, piercing tube 634 must be flexible enough to be delivered through vasculature to the treatment area white still rigid enough to pierce myocardial tissue.

Piercing tube 634 may include a stop collar (not shown) to aid in positioning distal anchor by controlling the depth of penetration of piercing tube 634 into the wall. A proximal portion of stop collar may be attached to the outside surface of a distal portion of piercing tube 634. One embodiment of a stop collar is cylindrical and has longitudinal slots spaced around a distant portion of the cylinder to form segments that are heat set or otherwise set such that they flare out away from the longitudinal axis of the cylinder when stop collar is released from guiding sheath 632.

Holding tube 636 and push cylinder 638 also comprise one or more biocompatible materials. Push cylinder 638 may be either a hollow or a solid elongated cylinder. Both holding tube 636 and push cylinder 638 must be flexible while still having sufficient rigidity to exert force on a heart chamber wall or an anchor, as described below.

Adjustment member 630 can be made from suitable biocompatible chord that can be routed through the eyelet at the end of the tension member such that both ends of the adjustment member extend tram the proximal end of the delivery catheter. During deployment of the device, the clinician can apply a slight tension to the adjustment member while pushing on the push cylinder to expel the device from the catheter. This allows the clinician to ensure that the proximal anchor will not be forced into the heart chamber that is being treated. Once the anchor has been properly deployed and the device is properly adjusted, the clinician can pull on one of the free ends of the adjustment member and withdraw the member through the eyelet and out of the delivery catheter.

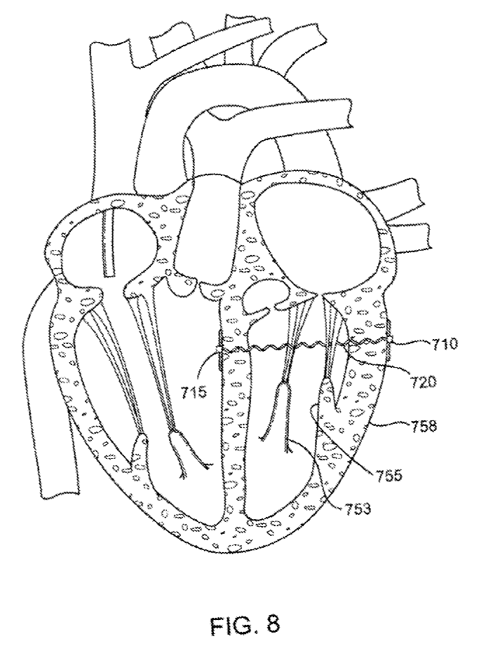

FIG. 7 shows a device for treating mitral valve regurgitation at an intermediate step in the deployment. FIG. 8 shows the device fully deployed, wherein the tension member 720 Is extended across a chamber of a heart, the proximal anchor 715 is deployed against a first wall of the heart chamber, and the distal anchor 710 is deployed against the exterior of a second wall of the heart chamber, The device depicted in FIGS. 7 & 8 is the device shown in FIGS. 1 & 2 and described above, chamber where the device is deployed is the left ventricle, the first walt is the septal wall between the right and left ventricles of the heart, and the second wall is the left ventricular free wall.

When positioned across a heart chamber, the anchors and tether are under continuously varying tension due to the motion of the beating heart. To withstand this environment, the tension member comprises an elastic, biocompatible, metallic or polymeric material that combines elasticity, flexibility, high strength, and high fatigue resistance. For example, the device may be formed using metallic wire, metallic tubes, polymer braid, polymer thread, elastomeric monofilament, elastomeric yam, etc, so long as the material has suitable elastic properties to allow the tension member to apply a continuous tension force between the two anchor members.

In some embodiments, an antithrombotic component may be included in the chemical composition of the material used to make the tensioning device, Alternatively, an elastomeric, polymeric, or metallic tether may be coated with a polymer that releases an anticoagulant and thereby reduces the risk of thrombus formation. If desired, additional therapeutic agents or combinations of agents may be use, including antibiotics and anti-inflammatory Other embodiments of the devices disclosed herein can include a coating or sleeve made from Dacron® fiber or the like.

To ensure proper positioning, it is desirable that tensioning device be visible using fluoroscopy, echocardiography, intravascular ultrasound, angioscopy, or another means of visualization. Some embodiments of the devices disclosed herein can be coated with echogenic materials and some devices can include materials having a high X-ray attenuation coefficient (radiopaque materials). The devices may be made in whole or in part from the material, or they may be coated in whole or in part by radiopaque materials. Alloys or plastics may include radiopaque components that are integral to the materials. Examples of suitable radiopaque material include, but are not limited to gold, tungsten, silver, tantalum, iridium, platinum, barium sulfate and bismuth sub-carbonate.

Referring again to FIG. 7, for delivery, the device is in a configuration similar to that shown in FIG. 3. Tensioning device is slidably received within a delivery catheter 730. The delivery catheter 730 has the same components of the catheter depicted in FIG. 3 and the delivery/positoning of the tensioning device shown in FIG. 7 will be described using the terms used to describe the components of the delivery catheter 330 shown in FIG. 3.

The arms of proximal anchor and distal anchor respectively, start in a folded, radially compressed configuration. Proximal anchor is positioned within the lumen of a holding tube while distal anchor is positioned within the lumen of a piercing tube. A push cylinder abuts the proximal and of proximal anchor. Holding tube abuts the proximal end of distal anchor.

Delivery catheter carrying tensioning device is passed through the venous system and into a patient's right ventricle. This may be accomplished as shown in FIG. 7, in which delivery catheter 730 has been inserted into either the jugular vein or the subolavian vein and passed through superior vena cava 742 into right atrium 744, and then passed through the tricuspid valve into right ventricle 748. Alternatively, the catheter may be inserted into the femoral vein and passed through the common iliac vein and the inferior vena cava into the right atrium, then through the tricuspid valve into the right ventricle. The procedure may be visualized using fluoroscopy, echocardiography, intravascular ultrasound, angioscopy, or other means of visualization.

The distal tip of delivery catheter 730 is positioned against the right ventricular surface of the septum 750. The delivery catheter then pierces the septal wall by extending piercing tube beyond the distal end of guiding sheath until the tube pierces through the septal wall.

The distal anchor 710 is then engaged with a second wall of the heart chamber. In the depicted embodiment, the distal anchor 710 is expanded on the exterior surface of the myocardium and engaged with the free wall of left ventricle 752. To accomplish this, piercing tube is advanced across the left ventricle between the papillary muscles and the chordae tendinae attached the mitral valve 756 leaflets that separate the left ventricle 752 and the left atrium 754. The piercing tube is allowed to pierce into the free wall of left ventricle 752 and the guiding sheath and its contents are advanced through the septal wall and across the left ventricle.

Distal anchor 710 is then pushed out of piercing tube using holding tube, at which time arms are permitted to expand away from the body of distal anchor and fix the distal anchor firmly against the exterior of the wall. In at least one embodiment of the current invention, the anchor remains inside the pericardial membrane but in other embodiments the anchor is outside of the pericardial membrane such that the membrane is between the deployed anchor and the myocardium. Alternatively, distal anchor 710 may be delivered by extending piercing tube to penetrate through the free wall of the left ventricle, and then retracting piercing tube while holding distal anchor stationary with holding tubes. Distal anchor is thus released from the distal end of piercing tube and permitted to self-expand.

Following deployment of the distal anchor, piercing tube is withdrawn across the left ventricle, through the septal wall, and into guiding sheath. The tension member, which links distal anchor with proximal anchor, is allowed to slide out transverse the left ventricle as piercing tube is withdrawn.

The proximal anchor is then deployed such that it engages with the septal wall. To accomplish this, proximal anchor is pushed out of holding tube using push cylinder, or holding tube is withdrawn while proximal anchor is maintained stationary with push cylinder. Proximal anchor is thus released from the distal end of piercing tube and permitted to if-expand as seen in FIGS. 1 & 2. During the deployment of the device the adjustment member can be used as described above to make sure that the proximal anchor is not extended into the left ventricle.

Referring to FIG. 8, the deployment of the device is complete, with the distal anchor 710 resting against the exterior of the free wall 758 and the proximal anchor 715 resting against the septal wail in the right ventricle. The tension member 720 extends across the left ventricle between the papillary muscles 755 & 753 and the chordae tendinae.

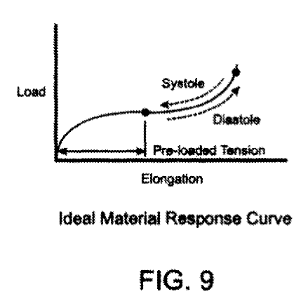

In order to resist excessive elongation during diastole, the materal used should stiffen dramatically when elongated. During systole, the tension member should again be elastic to as to recover or recoil. In some embodiments of the invention, it may be desirable to have some pre-load on the tension member so that the anchors remain seated and so that no slack develops in the tether. FIG. 9 shows an ideal response curve for the materials used to make tensioning devices of the current invention.

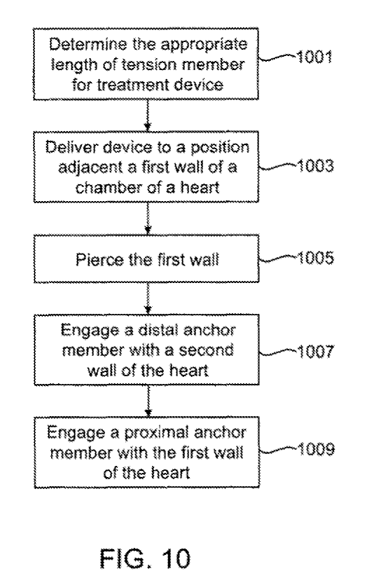

Referring to FIG. 10, a block diagram shows the steps of one method of using the devices disclosed herein. First, the treating clinician determines the desired wall tension of the heart, the length of device needed to appropriately reduce the distance between the septal wall and the free wall of the heart chamber, and the device characteristics as described above (block 1001). The device is then delivered to a position adjacent a first wall of the heart chamber (block 1002), which can be the septal wall as shown and described herein, or the device can also be delivered from the exterior of the heart using surgical techniques that are known in the art of cardiac surgery or even a catheter from a vessel that Is exterior to the chamber being altered. The fist wall is pierced (block 1005) and the second wall is pierced, so that a distal anchor can be engaged with the second wall (block 1007). A proximal anchor is then engaged with the first wall of the heart (block 11009) and the delivery device is removed. As a result of shortening the lateral space between the free wall and the septal wall, and reducing the wall tension, the geometry of the heart has been altered and mitral regurgitation is reduced.

The tension member is made from a material having suitable elasticity and constructed in a suitable configuration such that it applies a constant tension force between the two anchors to draw the walls of the left ventricle together and reduces both the radial tension on and the radial dimension of the mitral valve 754, thus improving coaption of the valve leaflets and reducing regurgitation. The tension member elongates and contracts (flexes) in response to a heartbeat when the anchors are secured and the tension member positioned across the heart chamber. This provides a shock absorbing effect that helps to protect the tensioning device from fatigue and reduces localized compressive pressure on tissue against which the anchors rest. In some embodiments of the invention, the anchors can also include flexing elements to further reduce fatigue and reduce pressure on the tissue.

The tensioning device may be placed In close proximity to the mitral valve, so that when the tension member contracts such that the distance between the proximal and distal anchors is adjusted, the outer cardiac wall is drawn toward the septal wall such that the anterior and posterior leaflets of the mitral valve are drawn together, thus reducing regurgitation, Alternatively, the device may be positioned across the left ventricle at an angle such that, for example, only one end of the device is anchored as close to the mitral valve annulus as possible.

Two or more devices may also be placed across the left ventricle in parallel, crisscrossing, or in other patterns as believed by the treating clinician to best achieve the desired result of radially compressing or relieving tension from the mitral valve. Alternatively, or in addition to ventricular placement, a tensioning device of the invention may be deployed across the left atrium, as approached from the right atrium, to radially compress or relieve tension from the mitral valve.

While several embodiments of the invention have been disclosed herein, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A device for implanting in a heart chamber to treat mitral valve regurgitation by reducing the lateral distance between the septum and the free wall of a the heart, the device comprising:
an elongated tension member having a long axis with a distal end and a proximal end, the tension member being elastic along the long axis such that when a force is applied, the tension member stretches from a first length to a second length and when the force is removed the tension member returns to the first length;
a distal anchor member attached to the distal end of the tension member; and
a proximal anchor member.

2. The device of claim 1 wherein when the device is positioned across a chamber of a heart, the length of the tension member changes in response to a heartbeat.

3. The device of claim 1 wherein the tension member is a helical extension spring.

4. The device of claim 3 wherein the tension member is made from a material selected from a group consisting of a nickel-titanium alloy, a nickel-cobalt alloy, a cobalt alloy, a thermoset plastic, a thermoplastics, stainless steel, a biocompatible shape-memory material, a biocompatible superelastic material, and a combination thereof.

5. The device of claim 1 wherein the tension member is made from an elastomeric material.

6. The device of claim 5 wherein the tension member is made from a material selected from a group consisting of silicone and urethane.

7. The device of claim 1 wherein at least one of the anchor members comprises a plurality of flexible arms.

8. The device of claim 1 wherein at least one of the anchor members comprises an expandable tubular braided portion.

9. The device of claim 1 wherein the anchor members can be compressed into a delivery configuration and placed within a lumen of a delivery catheter, and wherein the anchor members self-expand when they are released from the delivery catheter.

10. The device of claim 1 wherein the proximal anchor is adjustably attachable and the proximity of the anchors, one to the other can be adjusted.

11. The device of claim 1 wherein at least a portion of the device includes a therapeutic agent selected from a group consisting of an antithrombotic, an anticoagulant, an antibiotic, an anti-inflammatory, and a combination thereof.

12. A device for implanting in a heart chamber to treat mitral valve regurgitation by reducing the lateral distance between the septum and the free wall of a the heart, the device comprising:
an elongated tension member having a long axis with a distal end and a proximal end, the tension member being elastic along the long axis such that when a force is applied, the tension member stretches from a first length to a second length and when the force is removed the tension member returns to the first length;
a distal anchor member attached to the distal end of the tension member; and
a proximal anchor member attached to the proximal end of the tension member
whereby, when the device is positioned across a chamber of a heart, the length of the tension member changes in response to a heartbeat.

13. The device of claim 12 wherein the tension member is a helical extension spring made from a material selected from a group consisting of a nickel-titanium alloy, a nickel-cobalt alloy, a cobalt alloy, a thermoset plastic, a thermoplastic, stainless steel, a biocompatible shape-memory material, a biocompatible superelastic material, and a combination thereof.

14. The device of claim 12 wherein the tension member is made from an elastomeric material selected from a group consisting of silicone and urethane.

15. The device of claim 12 wherein at least one of the anchor members comprises a plurality of flexible arms; and
the anchor members can be compressed into a delivery configuration and placed within a lumen of a delivery catheter, and wherein the anchor members self-expand when they are released from the delivery catheter.

16. The device of claim 12 wherein at least one of the anchor members comprises an expandable tubular braided portion; and
the anchor members can be compressed into a delivery configuration and placed within a lumen of a delivery catheter, and wherein the anchor members self-expand when they are released from the delivery catheter.

17. The device of claim 12 wherein at least a portion of the device includes a therapeutic agent selected from a group consisting of an antithrombotic, an anticoagulant, an antibiotic, an anti-inflammatory, and a combination thereof.

18. A system for treating mitral valve regurgitation, comprising;
a deliver catheter; and
a tensioning device received in the delivery catheter, the tensioning device including an elongated tension member having a long axis with a distal end and a proximal end, the tension member being elastic along the long axis such that when a force is applied, the tension member stretches from a first length to a second length and when the force is removed the tension member returns to the first length, a distal anchor member attached to the distal end of the tension member, and a proximal anchor member; and
whereby the length of the tension member changes in response to a heartbeat when the device is positioned across a chamber of a heart.
